(19) United States
(12) Patent Application Publication
(10) Pub. No.: US 2023/0106528 A1
(43) Pub. Date: Apr. 6, 2023

(54) Modular Therapeutic Eye Mask

(71) Applicant: Selena Porter, Las Vegas, NV (US)

(72) Inventor: Selena Porter, Las Vegas, NV (US)

(21) Appl. No.: 17/959,110

(22) Filed: Oct. 3, 2022

Related U.S. Application Data
(63) Continuation-in-part of application No. 29/843,724, filed on Jun. 23, 2022.
(60) Provisional application No. 63/251,267, filed on Oct. 1, 2021.

(51) Int. Cl. A61F 7/02; A61F 9/04

(52) U.S. Cl. CPC A61F 7/02; A61F 9/04; A61F 2007/023; A61F 2007/0004

(57) ABSTRACT

A modular therapeutic eye mask is an apparatus that is used to help the user to sleep naturally and comfortably while receiving various therapeutic benefits. To do so, the apparatus includes a mask body, a support band, and a plurality of therapeutic inserts. The mask body is designed to be comfortably worn around the user's eyes to completely block any light from the surroundings. The mask body is also designed to retain the plurality of therapeutic inserts as well as other mask accessories that help the user to naturally sleep. The support band is designed to comfortably position the mask body and the plurality of therapeutic inserts retained within the mask body against the user's face. The plurality of therapeutic inserts includes several therapeutic inserts that provide various therapeutical benefits when at least one therapeutic insert is used with the mask body while the user is resting.

The current application is a continuation-in-part (CIP) application of the U.S. design application Ser. No. 29/843,724 filed on Jun. 23, 2022.

The current application also claims a priority to the U.S. provisional patent application Ser. No. 63/251,267 filed on Oct. 1, 2021. The current application is filed on Oct. 3, 2022, while Oct. 1, 2022 was on a weekend.

### FIELD OF THE INVENTION

The present invention relates generally to sleep accessories and therapeutic devices. More specifically, the present invention provides a modular eye mask that is more comfortable to wear to enable the user to receive various therapeutic benefits while wearing the eye mask.

### BACKGROUND OF THE INVENTION

People who have trouble sleeping at night are familiar with the effects caused by sleep deprivation. Some of the most common effects of sleep deprivation can be irritability, drowsiness, fatigue, among other things. Research has even found that sleep deprivation can promote the development of more serious issues such as high blood pressure and diabetes. Unfortunately, many environmental factures such as excess light in the room, mental or emotional issues, or medical ailments further prevent people from falling asleep naturally. To help with this issue, many sleep aid products have been made available, such as sleep eye masks. Traditional sleep eye masks are frequently used to block out excess light in the room, though these sleep masks do not offer much assistance for the other potential issues mentioned. Other sleep aid products include sleep medications or supplements such as melatonin that are available to help people sleep better. However, many people would prefer to fall asleep naturally and without the aid of medications or supplements. Therefore, there is a need for a sleep aid that helps users to naturally fall sleep in a comfortable and therapeutic manner.

An objective of the present invention is to provide a modular therapeutic eye mask that offers a solution to several issues faced by people with sleep deprivation. The present invention is designed to be comfortable to wear by the user to block light from the surroundings while the user is resting. Another objective of the present invention is to provide a modular therapeutic eyes mask that soothes and stimulates the user while resting. The present invention is designed to receive several therapeutic inserts that provide a range of therapeutic benefits including, but not limited to, a hot pack insert, a cold pack insert, or an aromatherapeutic insert. Further, the present invention is designed to strategically compress the user's head to help soothe headaches and to promote mentally stimulating thoughts. Additional features and benefits of the present invention are further discussed in the sections below.

### SUMMARY OF THE INVENTION

The present invention is a modular therapeutic eye mask designed to help the user to sleep better and naturally. The present invention includes a mask body designed to be comfortably worn by the user to completely cover the user's eyes so that the user can naturally fall asleep. The present invention also includes a support band that is preferably made from lace fabric so that the support band can comfortably keep the mask body positioned against the users' eyes. Further, the mask body is designed with added padding strategically positioned around the temple areas of the user's face to soothe the user while the user is resting.

Further, the mask body is designed to securely retain several therapeutic inserts that provide different therapeutic benefits to the user while the user is resting. The therapeutic inserts can include, but are not limited to, a hot pack insert, a cold pack insert, and/or an aromatherapeutic insert. Furthermore, the mask body is designed to receive different removable attachments that aid the user to sleep comfortably. For example, a pair of ear plugs can be provided that can be removably attached to the mask body so that the user can wear the ear plugs and the mask body at the same time while resting. Additional accessories can be provided that can be removably attached to the mask body to further aid the user to sleep comfortably and naturally.

### BRIEF DESCRIPTION OF THE DRAWINGS

FIG. 1 is a top-front-perspective view of the present invention.

FIG. 2 is a bottom-rear-perspective view of the present invention.

FIG. 3 is a front view of the present invention.

FIG. 4 is a vertical cross-sectional view of the present invention taken in the direction of line 4-4 in FIG. 3.

FIG. 5 is a vertical cross-sectional view of the present invention taken in the direction of line 5-5 in FIG. 3.

FIG. 6 is a front view of an electric heat pad from a plurality of therapeutic inserts of the present invention.

FIG. 7 is a front view of a reusable aromatherapy heat pack from the plurality of therapeutic inserts of the present invention.

FIG. 8 is a front view of a reusable cold pack from the plurality of therapeutic inserts of the present invention.

FIG. 9 is a front view of the present invention, wherein the electric heat pad is shown positioned within the mask body of the present invention.

FIG. 10 is a vertical cross-sectional view of the present invention taken in the direction of line 10-10 in FIG. 9.

FIG. 11 is a front view of a first strap, a second strap, a first ear plug, and a second ear plug of the present invention.

FIG. 12 is a front view of the present invention, wherein the first strap and the second strap are shown attached onto the mask body, wherein the first ear plug is shown connected to the first strap, and wherein the second ear plug is shown connected to the second strap.

### DETAIL DESCRIPTIONS OF THE INVENTION

All illustrations of the drawings are for the purpose of describing selected versions of the present invention and are not intended to limit the scope of the present invention.

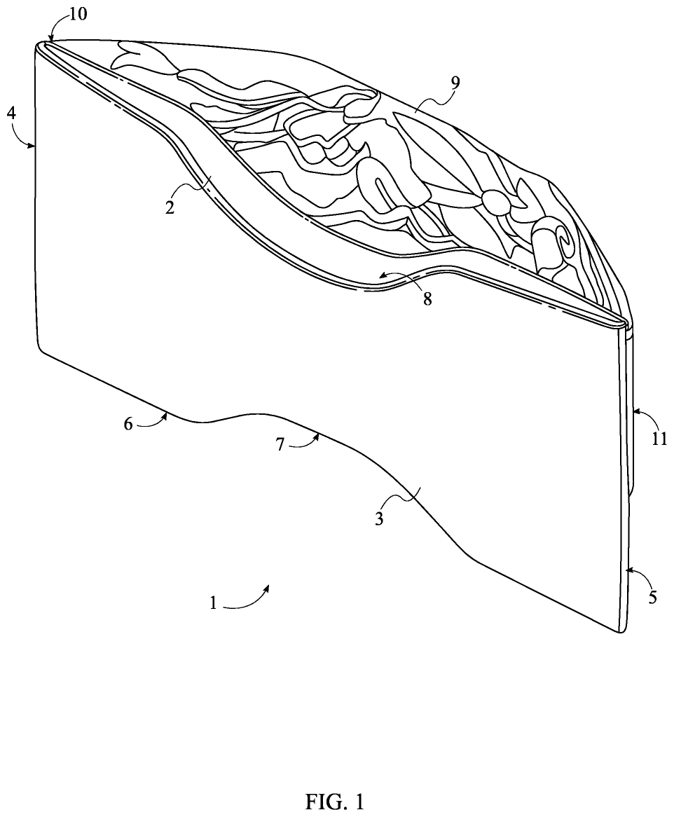

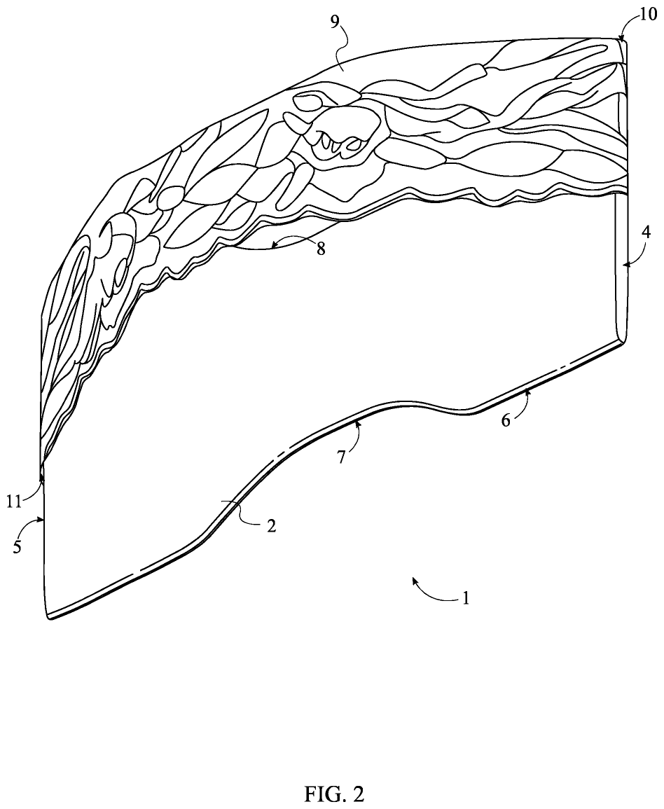

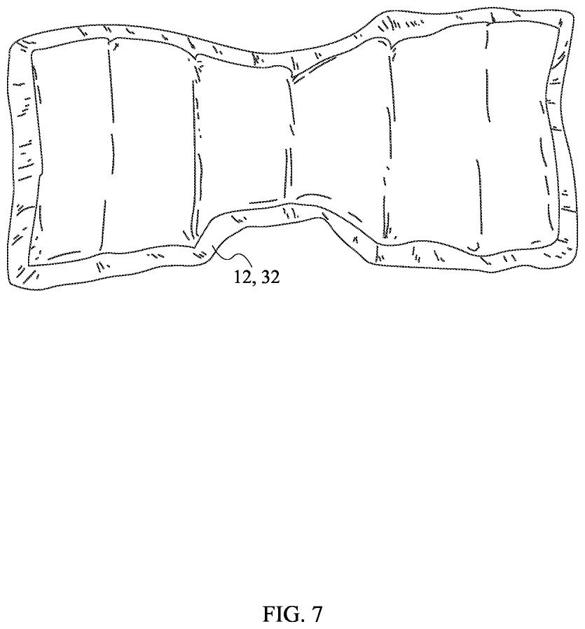

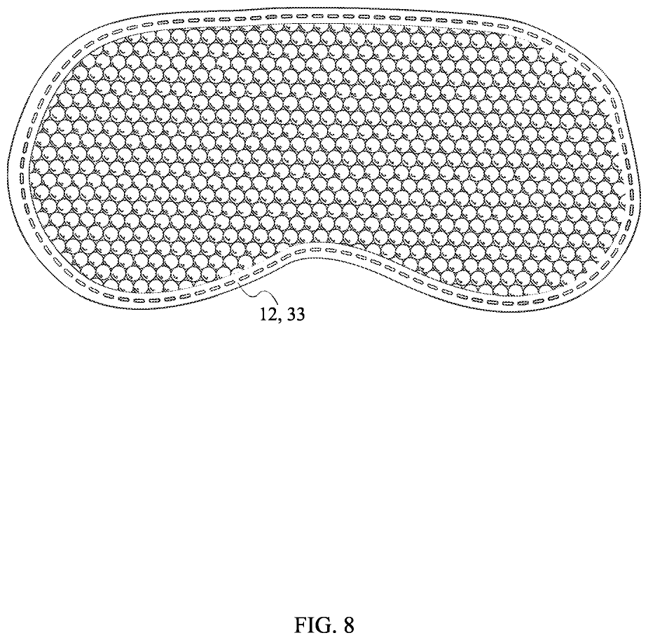

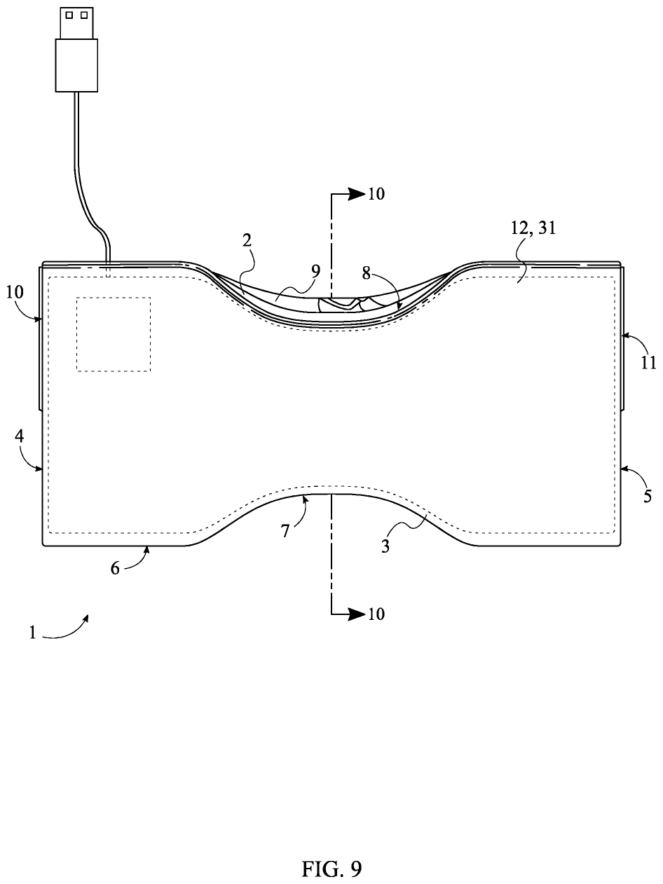

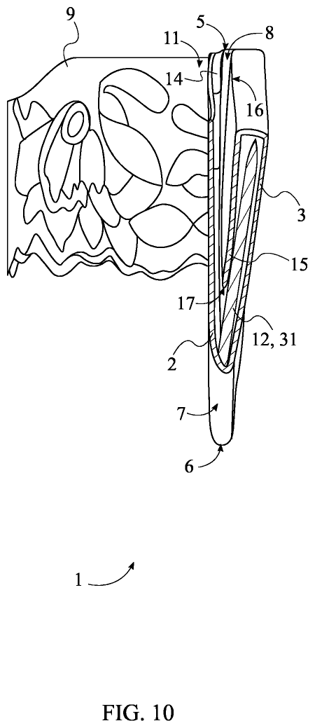

The present invention is a modular therapeutic eye mask that is used to help the user to sleep naturally and comfortably while receiving various therapeutic benefits. As can be seen in FIGS. 1, 2, and 6 through 10, the present invention comprises a mask body 1, a support band 9, and a plurality of therapeutic inserts 12. The mask body 1 is designed to be comfortably worn to completely block any light from the surroundings. The mask body 1 is also designed to retain the plurality of therapeutic inserts 12 as well as other accessories that help the user to sleep naturally. The support band 9 is designed to comfortably position the mask body 1 and the plurality of therapeutic inserts 12 retained within the mask body 1 against the user's face. The plurality of therapeutic inserts 12 includes several therapeutic inserts that provide various benefits when the therapeutic inserts are used with the mask body 1 while the user is resting.

The general configuration of the aforementioned components enables the user to naturally sleep while receiving desired therapeutic benefits. As can be seen in FIGS. 1, 2, and 6 through 10, the mask body 1 is preferably an overall thin, hollow, and soft structure designed to match the curvature of the user's face. Accordingly, the mask body 1 comprises a proximal mask panel 2, a distal mask panel 3, a first widthwise edge 4, a second widthwise edge 5, a lengthwise edge 6, and a lengthwise opening 8. The proximal mask panel 2 and the distal mask panel 3 are two thin panels made from soft material such as, but not limited to, fabric material, that are shaped to match the contours of the user's face. The first widthwise edge 4 and the second widthwise edge 5 preferably correspond to the shortest lateral edges of the mask body 1. Further, the lengthwise edge 6 corresponds to the longest closed edge of the mask body 1, while the lengthwise opening 8 corresponds to the longest open edge of the mask body 1 through which the plurality of therapeutic inserts 12 can be inserted into the mask body 1. Further, the support band 9 is an elongated, thin body with a width large enough to not apply too much pressure on the back of the user's head. Accordingly, the support band 9 comprises a first band end 10 and a second band end 11 corresponding to the terminal ends of the support band 9. In addition, the support band 9 is preferably made from a soft, ornamental material such as lace fabric which enables the support band 9 to have a unique ornamental design.

As can be seen in FIGS. 1, 2, and 6 through 10, to assemble the present invention, the first widthwise edge 4 and the second widthwise edge 5 are positioned opposite to each other about the mask body 1 due to the elongated shape of the mask body 1. The proximal mask panel 2, the distal mask panel 3, and the first band end 10 are connected along the first widthwise edge 4 to secure the proximal mask panel 2, the distal mask panel 3, and the support band 9 together along the first widthwise edge 4. Similarly, the proximal mask panel 2, the distal mask panel 3, and the second band end 11 are connected along the second widthwise edge 5 to secure the proximal mask panel 2, the distal mask panel 3, and the support band 9 together along the second widthwise edge 5. Further, the lengthwise opening 8 and the lengthwise edge 6 are positioned opposite to each other about the mask body 1. The proximal mask panel 2 and the distal mask panel 3 are also connected along the lengthwise edge 6 to secure the proximal mask panel 2 to the distal mask panel 3. This leaves the edge corresponding to the lengthwise opening 8 unconnected, which enables the user to selectively open and close the lengthwise opening 8 to insert a desired therapeutic insert from the plurality of therapeutic inserts 12. Finally, once the user has inserted a selected therapeutic insert through the lengthwise opening 8, the selected therapeutic insert from the plurality of therapeutic inserts 12 is positioned within the mask body 1.

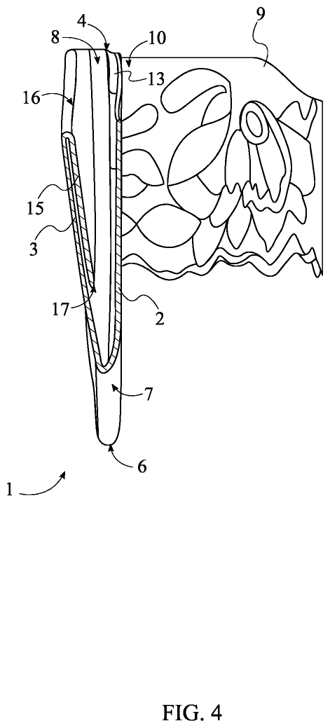

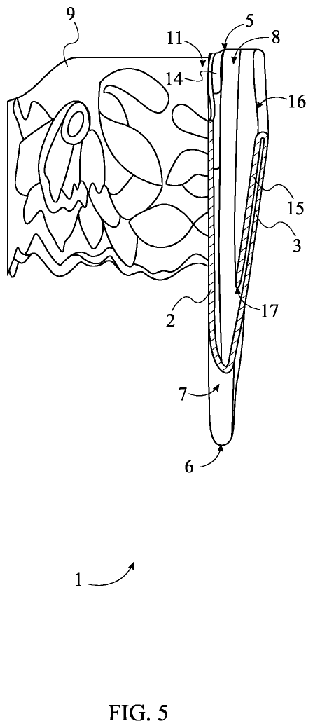

As previously discussed, the mask body 1 can be designed to match the physical features of the user's face so that the mask body 1 can be comfortably worn on the user's face for long periods of time. As can be seen in FIGS. 1 through 5, the lengthwise edge 6 may comprise a nose bridge recession 7 that accommodates the user's nose bridge for more comfort while wearing the mask body 1. In addition, the nose bridge recession 7 is centered along the lengthwise edge 6 to match the location of the user's nose. In other embodiments, the mask body 1 can be further tailored to accommodate other facial features or facial accessories.

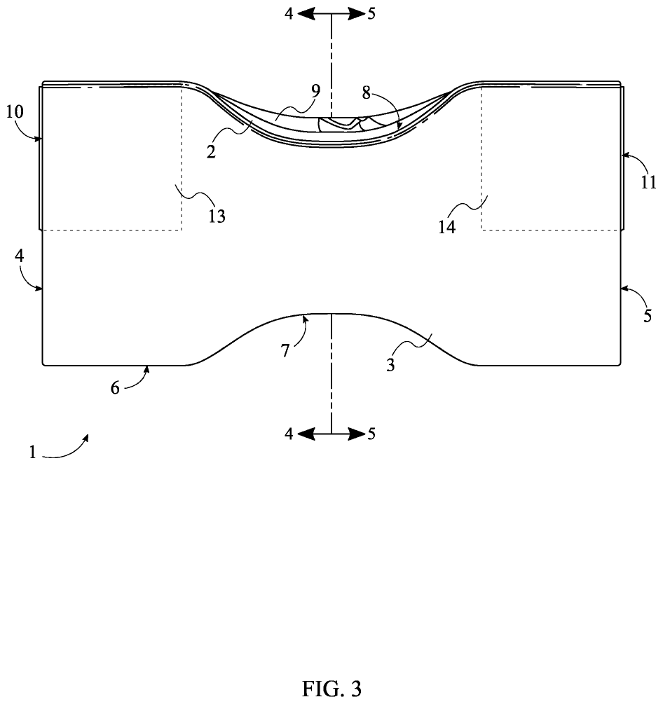

In addition to accommodating the facial features of the user, the mask body 1 can be designed to physically soothe the user when the user is wearing the present invention. As can be seen in FIGS. 3 through 5, the present invention may further comprise a first pad 13 and a second pad 14. The first pad 13 and the second pad 14 include padding material designed to softly apply pressure to the user's temples in order to soothe the user while the user is resting. Accordingly, the first pad 13 and the second pad 14 are positioned within the mask body 1 to hide the first pad 13 and the second pad 14 from view. The first pad 13 is positioned adjacent to the first widthwise edge 4. In addition, the first pad 13 is connected onto the proximal mask panel 2, adjacent to the lengthwise opening 8. Thus, the first pad 13 is positioned close to the corner between the lengthwise opening 8 and the first widthwise edge 4 so when the user is wearing the mask body 1, the first pad 13 is positioned against a temple to apply pressure. Similarly, the second pad 14 is positioned adjacent to the second widthwise edge 5. In addition, the second pad 14 is connected onto the proximal mask panel 2, adjacent to the lengthwise opening 8. Thus, the second pad 14 is positioned close to the opposite corner between the lengthwise opening 8 and the first widthwise edge 4 so when the user is wearing the mask body 1, the first pad 13 is positioned against the other temple to apply pressure. In other embodiments, additional padding can be provided throughout the mask body 1 to apply pressure on different areas of the user's face.

As can be seen in FIGS. 3 through 5, 9, and 10, the prevent the selected therapeutic insert from the plurality of therapeutic inserts 12 from falling off the mask body 1 through the lengthwise opening 8 while the user is resting, the present invention may further comprise a closure flap 15. The closure flap 15 is designed to close the lengthwise opening 8 when the selected therapeutic insert is positioned within the mask body 1. The closure flap 15 also keeps the lengthwise opening 8 closed when no therapeutic insert has been inserted into the mask body 1 to prevent dirt or other foreign objects to enter the mask body 1. To do so, the closure flap 15 is preferably a thin piece of material similar to the material of mask body 1, such as fabric material. The closure flap 15 comprises a flap fixed edge 16 and a flap free edge 17, where the flap fixed edge 16 corresponds to the edge that connects the closure flap 15 to the mask body 1, and the flap free edge 17 corresponds to the loose edge of the closure flap 15. Accordingly, the closure flap 15 is positioned within the mask body 1 adjacent to the lengthwise opening 8 to close the lengthwise opening 8. The flap fixed edge 16 and the flap free edge 17 are also positioned opposite to each other across the closure flap 15 due to the length of the closure flap 15. Further, the flap fixed edge 16 is connected onto the distal mask panel 3, adjacent to the lengthwise opening 8, to secure the closure flap 15 to the distal mask panel 3. This way, the closure flap 15 does not alter the shape of the mask body 1 while still being able to close the lengthwise opening 8.

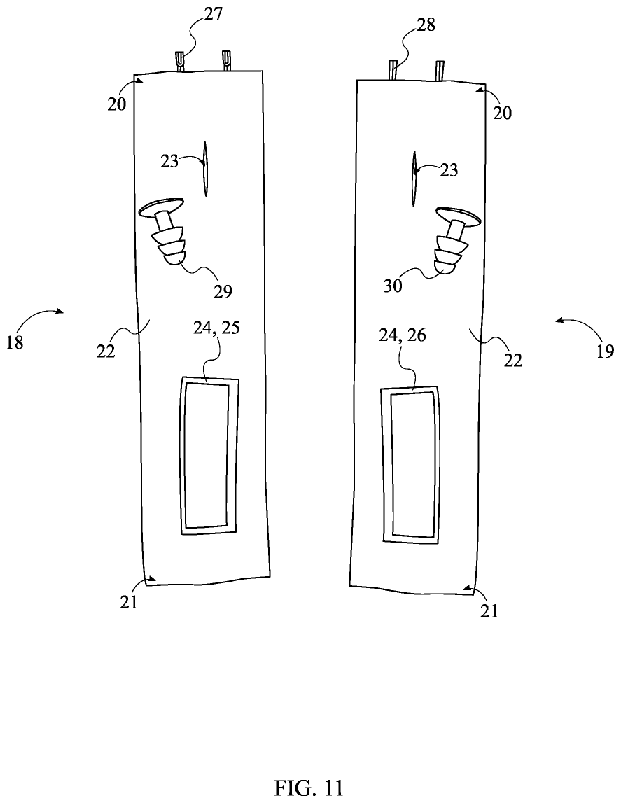

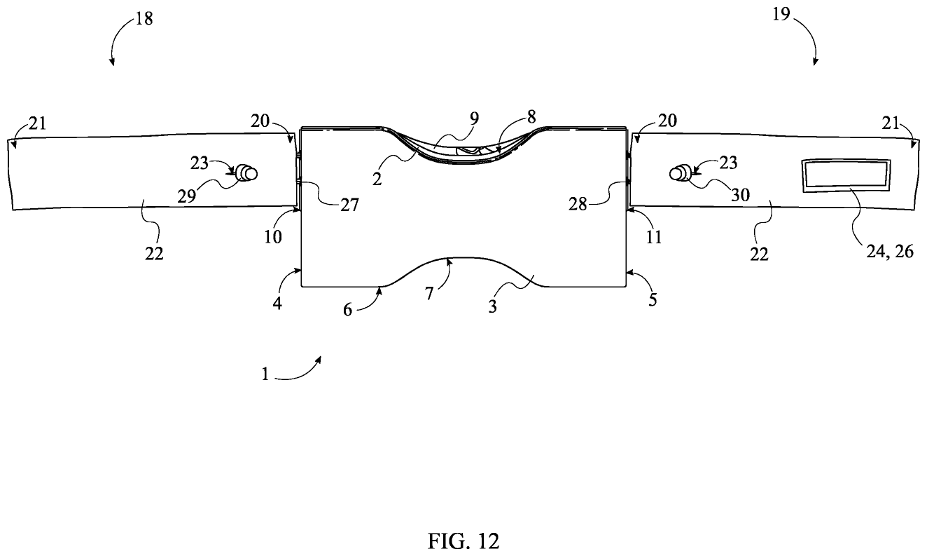

In some embodiments, the present invention can be configured to accommodate different accessories to help the user sleep naturally. For example, the present invention can include ear plugs to block noise from the surroundings. As can be seen in FIGS. 11 and 12, to accommodate these accessories, the present invention may further comprise a first strap 18, a second strap 19, and a strap fastening mechanism 24. The first strap 18 and the second strap 19 accommodate the different accessories in such a way that the accessories are secured to the mask body 1. This prevents the accessories from coming loose while the user is resting. The strap fastening mechanism 24 enables the fastening of the first strap 18 to the second strap 19 so that the first strap 18 and the second strap 19 can be used instead of the support band 9. Further, the first strap 18 and the second strap 19 each comprises an anterior strap end 20 and a posterior strap end 21 corresponding to the terminal ends of each strap. In addition, the strap fastening mechanism 24 comprises a first interlocking piece 25 and a second interlocking piece 26 that enable the selective fastening and unfastening of the strap fastening mechanism 24. For example, the strap fastening mechanism 24 can be a hook and loop fastener, with the first interlocking piece 25 corresponding to the hook portion of the fastener, and the second interlocking piece 26 corresponding to the loop portion of the fastener. Accordingly, the anterior strap end 20 of the first strap 18 is attached onto the first widthwise edge 4, adjacent to the lengthwise opening 8. This way, the first strap 18 can be removably attached to the mask body 1. Similarly, the anterior strap end 20 of the second strap 19 is attached onto the second widthwise edge 5, adjacent to the lengthwise opening 8. This way, the second strap 19 can also be removably attached to the mask body 1. Furthermore, the first interlocking piece 25 is connected onto the posterior strap end 21 of the first strap 18 to secure the first interlocking piece 25 to the first strap 18. Similarly, the second interlocking piece 26 is connected onto the posterior strap end 21 of the second strap 19 to secure the second interlocking piece 26 to the second strap 19. Then, to fasten the first strap 18 to the second strap 19, the user just engages the first interlocking piece 25 with the second interlocking piece 26.

As previously discussed, both the first strap 18 and the second strap 19 can be removably attached to the mask body 1. As can be seen in FIGS. 11 and 12, to enable the removable attachment of both the first strap 18 and the second strap 19 to the mask body 1, the present invention may further comprise a first hook-eyelet fastener 27 and a second hook-eyelet fastener 28. The first hook-eyelet fastener 27 and the second hook-eyelet fastener 28 enable the removable attachment of the first strap 18 and the second strap 19 to the mask body 1, respectively. To do so, the anterior strap end 20 of the first strap 18 is attached onto the first widthwise edge 4 by the first hook-eyelet fastener 27. For example, the eyelet portion of the fastener can be connected to the first lengthwise edge 6, while the hook portion of the fastener is connected to the anterior strap end 20 of the first strap 18. Likewise, the anterior strap end 20 of the second strap 19 is attached onto the second widthwise edge 5 by the second hook-eyelet fastener 28. For example, the eyelet portion of the fastener can be connected to the second lengthwise edge 6, while the hook portion of the fastener is connected to the anterior strap end 20 of the second strap 19. This way, the user can choose to use the first strap 18 and the second strap 19 if desired by easily attaching the first strap 18 and the second strap 19 to the mask body 1 using the corresponding hook-eyelet fastener.

Further, as previously discussed, the present invention can include means to block noise from the surroundings to help the user rest comfortably. As can be seen in FIGS. 11 and 12, the present invention may further comprise a first ear plug 29 and a second ear plug 30. The first ear plug 29 and the second ear plug 30 can be used to plug the user's ear so that the user is not disturbed by any noise while resting. To secure the ear plugs to the corresponding straps, the first strap 18 and the second strap 19 each further comprises a strap body 22 and a plug-receiving hole 23. The plug-receiving hole 23 enables each ear plug to be secured to the strap body 22 of the corresponding strap. For example, each ear plug can include a plug body and a plug connector. The plug body preferably corresponds to the part of the ear plug that is inserted into the ear, while the plug connector corresponds to the part of the ear plug that is inserted into the plug-receiving hole 23. The plug connector can include, but is not limited to, a button that fits into the plug-receiving hole 23. Accordingly, the plug-receiving hole 23 laterally traverses through the strap body 22 to enable the corresponding ear plug to be secured to the strap body 22. The plug-receiving hole 23 is positioned offset from the anterior strap end 20 at a predetermined distance. In addition, the plug-receiving hole 23 is also positioned offset from the posterior strap end 21 at a predetermined distance. This way, the plug-receiving hole 23 can be positioned along the strap body 22 to match the position of the corresponding ear. Further, the first ear plug 29 is engaged into the plug-receiving hole 23 of the first strap 18, while the second ear plug 30 is engaged into the plug-receiving hole 23 of the second strap 19. Thus, once the user secures the first strap 18 and the second strap 19 to the mask body 1, the user can insert the first ear plug 29 and the second ear plug 30 into the plug-receiving hole 23 of the first strap 18 and the plug-receiving hole 23 of the second strap 19, respectively. The user then inserts the first ear plug 29 and the second ear plug 30 into the user's ears. Then, the user wraps the first strap 18 and the second strap 19 around the back of the user's head to engage the strap fastening mechanism 24 which secures the first strap 18, the second strap 19, and the mask body 1 in position. In other embodiments, other accessories can be provided that can be removably secured to the mask body 1.

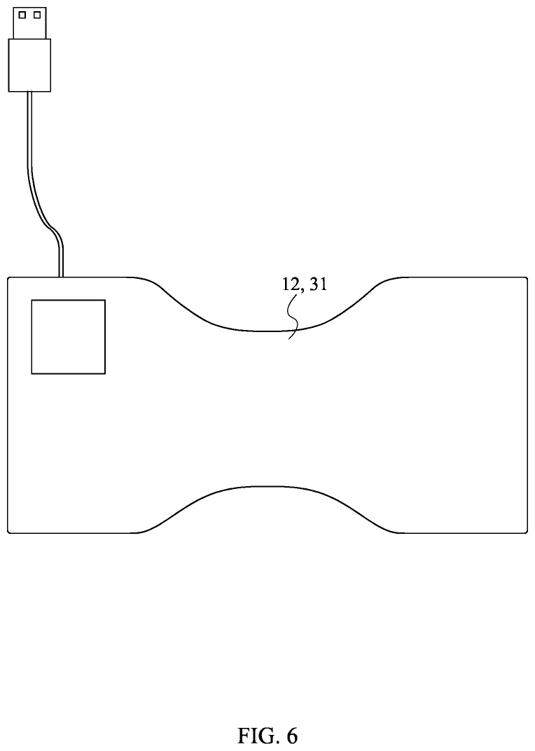

As previously discussed, the plurality of therapeutic inserts 12 includes various therapeutic inserts that provide different therapeutic benefits to the user while the user is wearing the mask body 1. As can be seen in FIGS. 6 through 10, each therapeutic insert is shaped and sized to match the shape and size of the mask body 1 so that the selected therapeutic insert can fit inside the mask body 1. In one embodiment, a therapeutic insert of the plurality of therapeutic inserts 12 can be an electric heat pad 31. The electric heat pad 31 is a heat pad designed to match the shape and size of the mask body 1 so that the electric heat pad 31 can be inserted into the mask body 1 through the lengthwise opening 8. The electric heat pad 31 can be connected to an external power source through a power connector including, but not limited to, a Universal Serial Bus (USB) connector that can be plugged into a USB port to power the heat pad. The heat pad can include several heat elements that are electrically powered. The electric heat pad 31 may also include a controller that enables the user to control the operation of the electric heat pad 31. Further, the electric heat pad 31 may include a power storage mechanism, such as rechargeable batteries, which enables the user to power the electric heat pad 31 without connected the electric heat pad 31 to an external power source.

As can be seen in FIGS. 6 through 10, in another embodiment, another therapeutic insert of the plurality of therapeutic inserts 12 can be a reusable aromatherapy heat pack 32. The reusable aromatherapy heat pack 32 includes several heat packs containing essential oils or aromatic compounds that when heated release the corresponding aromas to the surroundings. The reusable aromatherapy heat pack 32 can be heated up using external heat sources including, but not limited to, a microwave. So, once the reusable aromatherapy heat pack 32 is warmed up to a safe temperature, the user inserts the reusable aromatherapy heat pack 32 into the mask body 1. This way, when the user is wearing the mask body 1 with the reusable aromatherapy heat pack 32, the therapeutical aromas reach the user's face. In another embodiment, another therapeutic insert of the plurality of therapeutic inserts 12 can be a reusable cold pack 33. The reusable cold pack 33 can include several packets of freezable material that can be repeatedly frozen for several uses. The reusable cold pack 33 can be frozen using different heat exchanging mechanisms such as, but not limited to, a freezer. Thus, once the reusable cold pack 33 has been frozen or cooled down to a safe temperature, the user inserts the reusable cold pack 33 into the mask body 1. This way, when the user is wearing the mask body 1 with the reusable cold pack 33, the user's face is cooled down for various therapeutical purposes, such as reducing swelling or rejuvenating the user's facial skin. In other embodiments, additional therapeutical inserts can be included that provide different therapeutical benefits to the user.

Although the invention has been explained in relation to its preferred embodiment, it is to be understood that many other possible modifications and variations can be made without departing from the spirit and scope of the invention.

What is claimed is:

1. A modular therapeutic eye mask comprising:
a mask body;
a support band;
a plurality of therapeutic inserts;
the mask body comprising a proximal mask panel, a distal mask panel, a first widthwise edge, a second widthwise edge, a lengthwise edge, and a lengthwise opening;
the support band comprising a first band end and a second band end;
the first widthwise edge and the second widthwise edge being positioned opposite to each other about the mask body;
the proximal mask panel, the distal mask panel, and the first band end being connected along the first widthwise edge;
the proximal mask panel, the distal mask panel, and the second band end being connected along the second widthwise edge;
the lengthwise opening and the lengthwise edge being positioned opposite to each other about the mask body;
the proximal mask panel and the distal mask panel being connected along the lengthwise edge; and
a selected therapeutic insert from the plurality of therapeutic inserts being positioned within the mask body.

2. The modular therapeutic eye mask as claimed in claim 1, wherein the support band is made from lace fabric.

3. The modular therapeutic eye mask as claimed in claim 1 comprising:
the lengthwise edge comprising a nose bridge recession; and
the nose bridge recession being centered along the lengthwise edge.

4. The modular therapeutic eye mask as claimed in claim 1 comprising:
a first pad;
a second pad;
the first pad and the second pad being positioned within the mask body;
the first pad being positioned adjacent to the first widthwise edge;
the first pad being connected onto the proximal mask panel, adjacent to the lengthwise opening;
the second pad being positioned adjacent to the second widthwise edge; and
the second pad being connected onto the proximal mask panel, adjacent to the lengthwise opening.

5. The modular therapeutic eye mask as claimed in claim 1 comprising:
a closure flap;
the closure flap comprising a flap fixed edge and a flap free edge;
the closure flap being positioned within the mask body;
the flap fixed edge and the flap free edge being positioned opposite to each other across the closure flap; and
the flap fixed edge being connected onto the distal mask panel, adjacent to the lengthwise opening.

6. The modular therapeutic eye mask as claimed in claim 1 comprising:
a first strap;
a second strap;
a strap fastening mechanism;
the first strap and the second strap each comprising an anterior strap end and a posterior strap end;
the strap fastening mechanism comprising a first interlocking piece and a second interlocking piece;
the anterior strap end of the first strap being attached onto the first widthwise edge, adjacent to the lengthwise opening;
the anterior strap end of the second strap being attached onto the second widthwise edge, adjacent to the lengthwise opening;
the first interlocking piece being connected onto the posterior strap end of the first strap; and
the second interlocking piece being connected onto the posterior strap end of the second strap.

7. The modular therapeutic eye mask as claimed in claim 6 comprising:
a first hook-eyelet fastener;
a second hook-eyelet fastener;
the anterior strap end of the first strap being attached onto the first widthwise edge by the first hook-eyelet fastener; and
the anterior strap end of the second strap being attached onto the second widthwise edge by the second hook-eyelet fastener.

8. The modular therapeutic eye mask as claimed in claim 6 comprising:
a first ear plug;
a second ear plug;
the first strap and the second strap each further comprising a strap body and a plug-receiving hole;
the plug-receiving hole laterally traversing through the strap body;
the plug-receiving hole being positioned offset from the anterior strap end;
the plug-receiving hole being positioned offset from the posterior strap end;
the first ear plug being engaged into the plug-receiving hole of the first strap; and
the second ear plug being engaged into the plug-receiving hole of the second strap.

9. The modular therapeutic eye mask as claimed in claim 1, wherein a therapeutic insert of the plurality of therapeutic inserts is an electric heat pad.

10. The modular therapeutic eye mask as claimed in claim 1, wherein a therapeutic insert of the plurality of therapeutic inserts is a reusable aromatherapy heat pack.

11. The modular therapeutic eye mask as claimed in claim 1, wherein a therapeutic insert of the plurality of therapeutic inserts is a reusable cold pack.

12. A modular therapeutic eye mask comprising:
a mask body;
a support band;
a plurality of therapeutic inserts;
the mask body comprising a proximal mask panel, a distal mask panel, a first widthwise edge, a second widthwise edge, a lengthwise edge, and a lengthwise opening;
the support band comprising a first band end and a second band end;
the lengthwise edge comprising a nose bridge recession;
the first widthwise edge and the second widthwise edge being positioned opposite to each other about the mask body;
the proximal mask panel, the distal mask panel, and the first band end being connected along the first widthwise edge;
the proximal mask panel, the distal mask panel, and the second band end being connected along the second widthwise edge;
the lengthwise opening and the lengthwise edge being positioned opposite to each other about the mask body;
the proximal mask panel and the distal mask panel being connected along the lengthwise edge;
a selected therapeutic insert from the plurality of therapeutic inserts being positioned within the mask body;
the nose bridge recession being centered along the lengthwise edge; and
the support band being made from lace fabric.

13. The modular therapeutic eye mask as claimed in claim 12 comprising:
a first pad;
a second pad;
the first pad and the second pad being positioned within the mask body;
the first pad being positioned adjacent to the first widthwise edge;
the first pad being connected onto the proximal mask panel, adjacent to the lengthwise opening;
the second pad being positioned adjacent to the second widthwise edge; and
the second pad being connected onto the proximal mask panel, adjacent to the lengthwise opening.

14. The modular therapeutic eye mask as claimed in claim 12 comprising:
a closure flap;
the closure flap comprising a flap fixed edge and a flap free edge;
the closure flap being positioned within the mask body;
the flap fixed edge and the flap free edge being positioned opposite to each other across the closure flap; and
the flap fixed edge being connected onto the distal mask panel, adjacent to the lengthwise opening.

15. The modular therapeutic eye mask as claimed in claim 12 comprising:
a first strap;
a second strap;
a strap fastening mechanism;
the first strap and the second strap each comprising an anterior strap end and a posterior strap end;
the strap fastening mechanism comprising a first interlocking piece and a second interlocking piece;
the anterior strap end of the first strap being attached onto the first widthwise edge, adjacent to the lengthwise opening;
the anterior strap end of the second strap being attached onto the second widthwise edge, adjacent to the lengthwise opening;
the first interlocking piece being connected onto the posterior strap end of the first strap; and
the second interlocking piece being connected onto the posterior strap end of the second strap.

16. The modular therapeutic eye mask as claimed in claim 15 comprising:
a first hook-eyelet fastener;
a second hook-eyelet fastener;
the anterior strap end of the first strap being attached onto the first widthwise edge by the first hook-eyelet fastener; and
the anterior strap end of the second strap being attached onto the second widthwise edge by the second hook-eyelet fastener.

17. The modular therapeutic eye mask as claimed in claim 15 comprising:
a first ear plug;
a second ear plug;
the first strap and the second strap each further comprising a strap body and a plug-receiving hole;
the plug-receiving hole laterally traversing through the strap body;
the plug-receiving hole being positioned offset from the anterior strap end;
the plug-receiving hole being positioned offset from the posterior strap end;
the first ear plug being engaged into the plug-receiving hole of the first strap; and
the second ear plug being engaged into the plug-receiving hole of the second strap.

18. The modular therapeutic eye mask as claimed in claim 12, wherein a therapeutic insert of the plurality of therapeutic inserts is an electric heat pad.

19. The modular therapeutic eye mask as claimed in claim 12, wherein a therapeutic insert of the plurality of therapeutic inserts is a reusable aromatherapy heat pack.

20. The modular therapeutic eye mask as claimed in claim 12, wherein a therapeutic insert of the plurality of therapeutic inserts is a reusable cold pack.
